(19) United States
(12) Patent Application Publication
(10) Pub. No.: US 2008/0026373 A1
(43) Pub. Date: Jan. 31, 2008

(54) Assays Based On Light Emission From Analyte Complexes Within A Cassette

(76) Inventor: Natalia A. Rodionova, Waltham, MA (US)

Correspondence Address: FISH & RICHARDSON PC, P.O. BOX 1022, MINNEAPOLIS, MN, 55440-1022, US

(21) Appl. No.: 11/460,238

(22) Filed: Jul. 26, 2006

(51) Int. Cl. C12Q 1/68; C12M 3/00

(52) U.S. Cl. 435/6; 435/287.2

(57) ABSTRACT

Assays based on probes attached to surfaces enclosed within cassettes and cassettes and reading stations for the assays. After liquids flow over the probe or probe array to form an array of photo-emissive analyte complexes, and prior to reading, (a) liquid is removed by flow, and (b) a drying gas stream, is forced into the cassette and over the complexes for a drying interval to remove liquid residue. Heating assists the drying. Light from the analyte complexes is then read through a window of the cassette. The interval of drying may be of the order of about one minute. During a preceding wash phase, gas flow bursts through the gas inlet channel purge liquid contaminant. The probes e.g. may be oligonucleotides, peptides, polypeptides, proteins, antibodies, or small molecules (steroids, expression regulators, e.g. siRNA, or other ligands). A cassette has a passage leading from a bubble removal system to the probe-bearing surface and the desiccating gas stream is introduced to that passage. For wide arrays the common passage connects through a widening transition to a wide reaction chamber. A reader station includes an air pump and liquid pumping devices such as linear actuators to deflect liquid-pumping diaphragms of the cassette.

### TECHNICAL FIELD

This invention relates to liquid-based assays conducted by use of cassettes. In particular it relates to assays of the type based on detection of light passing through a cassette window from an analyte complex on a surface enclosed within a cassette.

### BACKGROUND

Assays between surface-attached binding agents or probes (receptor probes) and target molecules (ligands) in liquid solution are useful in many ways. In the biological field they are useful to detect the presence of particular biopolymers. The surface-attached probes may be oligonucleotides, peptides, polypeptides, proteins, antibodies or other molecules capable of binding with target molecules in the liquid solution. Arrays of probes for simultaneously conducting multiple assays are especially effective. The solutions containing the analyte may be blood or other body fluids, cell lysate, etc. The binding interactions are the basis for many methods and devices useful in a variety of applications, e.g., in diagnostics and other clinical work, in proteomics and in genomics. Useful techniques include ELISA, sandwich assays, competition assays, enzyme assays, sequencing by hybridization, SNP detection, differential gene expression analysis, identification of novel genes, gene mapping, etc.

One typical assay method involves biopolymeric probes immobilized in a two-dimensional array on a planar surface of a substrate on glass or the like to provide an array assembly. The array of probes may be produced by synthesis or spotting. A liquid solution containing or suspected of containing analytes that bind with the attached probes is brought into contact with the array or a portion of the array assembly. In many instances, a second member is positioned over and spaced from the array, the separation forming an assay region through which liquid can flow and in which the assaying can take place. The enclosed region is sometimes referred to as a “reaction chamber”, and the assembly is sometimes referred to as a “biochip” or “lab-on-a-chip”.

Usually, the targets in the liquid solution, if present, bind to the complementary probes on the substrate, each forming a binding complex. The binding of target molecules to probe features or spots is in a pattern of known locations. This provides desired information about the sample. In most instances, the target molecules are labeled with a detectable tag such as a fluorescent or luminescent label. The tags or other constituents of the bound complexes attain light-emissive states e.g., by exposure to light, by electric stimulation, by chemical reaction or by electro-stimulation of chemical reaction. The resultant complexes of binding pairs are then detected by optical means, e.g. the pattern of light is imaged with a focal plane camera such as a CCD camera or scanned for computer analysis.

For example, suitably filtered light from an LED or incandescent source or light from a laser, or an electric potential applied by a conductor, may be used to excite fluorescent tags, to generate a light signal only in those locations on a biochip that have probe molecules to which target molecules with light-emissive tags are bound. This pattern may then be optically detected.

Traditional processing of microarray bioassays has followed two paths. In a “manual” or “bench” process requiring presence of a skilled operator, the array typically has been processed and examined in the open. In a fully or partly automated process, in which an operator is excluded from important steps, the substrate bearing the array is imbedded in a cassette, with the array enclosed. Processing has then been conducted within the cassette and the resultant binding interaction or analyte complex has been detected while the array still remains enclosed.

For optical detection of a light pattern from a surface enclosed within a cassette, it is necessary that material forming the reaction chamber be at least partly light-transmissive to permit light of relevant wavelength to pass for detection. Where optical stimulation is employed, both stimulating and detection wavelengths must be transmitted through material of the cassette, though the light may be transmitted along different paths. With electroluminescence in which an electrical signal stimulates light emission from analyte complexes, or with chemiluminescence based on chemical interaction, the detection wavelength must be transmitted through material of the cassette.

The major benefit of the cassette-based assay is the exclusion of the human operator. All processing steps can be automated, providing standard conditions for comparison. Optical inspection of arrays enclosed in cassettes has been performed by keeping the assay wet during optical detection.

Reading while wet has been an accepted practice, long known. Automated protein or particle detection by fluorescent tagging, Flow Cytometry, is a wet process as the name indicates and detection has been in the wet state, Shapiro, H., Practical Flow Cytometry, (3^{rd}), Wiley LISS, 1995. Flow cytometry preceded the introduction of the technology of fluorescence detection of arrays, which borrowed the detection techniques and became known as “imaging cytometry.” Assay detection has also been based on fluorescence using evanescence surface waves to excite the attached probes, a technology performed in a liquid-filled reaction chamber. This is exemplified by Attrige, U.S. Pat. Nos. 5,369,717 and 5,166,515.

### SUMMARY OF INVENTION

I observed that measurements of optical signal level and signal-to-noise ratio (S/N ratio) obtained on microscope slides from the same batch differ greatly (often by a factor of 10 or more) between manual assays in which the microarray is in the open during detection versus the same assay in which the microarray remains enclosed within a cassette during detection. I have realized that it is possible to raise the signal-to-noise ratio of an assay system enclosed within a cassette by an order of magnitude or better by drying the enclosed array and evaluating the assay in situ in a well-dried condition as compared with the same assay in a wet condition or when liquid has been removed by flow from the cassette but the array has not been fully dried. This has significant consequences regarding the quantitative accuracy of assays and hence the scope of their usefulness.

To investigate the initially observed discrepancy, cassette-processed assays were measured in situ, wet, enclosed in a cassette, and later the window was removed, exposing the slide to drying, and the dried chip was again measured. It was noted that in order to achieve the same signal level of detected image, exposure for detection needed to be reduced from 6 seconds to 1 second approximately, from wet to dry condition.

In order to further explore these phenomena, a cassette simulation was built and processed manually, but maintained as a cassette. This was a 0.10 mm deep×4 mm wide×15 mm long reaction chamber, RC, formed over a microscope slide bearing a two dimensional array of probes and complexed fluorescently tagged analyte. The wall of the RC chamber opposite from the assay array was a transparent slide cover separated from the microscopic slide and assembled with a double sided adhesive tape. Both the slide and the slide cover and tape were the same as used to form the RC of a conventional closed cassette.

Measurements were taken through the transparent slide cover on an Axon confocal scanner. Measurements were taken in a variety of conditions: (1) Array as processed, wet; (2) same as (1) but with liquid removed by flow from the reaction chamber and replaced with air; (3) same as (2) but with a stream of air at 37 deg. C. pumped through the reaction chamber for a sustained period following removal of the liquid.

It is noted that removing the liquid by flow, i.e., simply replacing the liquid with air, did not alter the signal or the S/N ratio. Passing a stream of air through the cassette for a sustained period was, however, found to substantially improve both the signal and the S/N ratio as exhibited in the Figures. In the arrangement employed, signal and S/N ratio progressively increased with duration of the air stream through the cassette over an initial period of about two minutes.

The reaction chamber was than refilled with liquid buffer and it was observed that both the signal and S/N ratio reverted to the level of the original wet levels. This reversal—the degradation—was extremely fast.

The cycle was repeated a number of times with equal results.

The air flow was measured at approximately 250 cc per minute with a pressure drop of approximately 75 mbar and the air was heated to 37 deg. C.

Similar results were obtained with a fully implemented cassette having a reaction chamber with an intake flow cross section of 4 mm×0.1 mm, width and depth, the chamber being 12.5 mm in length. It had a supply channel with 0.5 mm×0.5 mm flow cross section. The reaction chamber opened to a wide waste storage volume equipped with a hydrophobic vent plug from the Porex Co., Fairburn, Ga., 30213-2828, exhibiting no appreciable air flow restriction. The pressure at the reaction chamber intake channel was approximately 100 mbar. The air pump employed was the NMP05M model from KNF Neuberger, Inc., Two Black Forest Road, Trenton, N.J. 08691. The pump is rated by the manufacturer for flow of about 250 cc/min. Other experiments were performed where the reaction chamber was heated from 37 to 44 Deg. C. The data are presented in the Figures.

The improved efficiency of the fluorescent tags provided by drying the enclosed complexes has been observed with tags of both Cy3 and Alexa that can be obtained from Molecular Probes, a division of Invitrogen, Carlsbad, Calif.

There are a number of aspects of invention.

According to one aspect of invention, a method is provided of conducting an assay by employing a cassette which encloses a surface to which at least one probe is attached, the surface associated with a liquid passage that enables liquid flow over the probe, the assay being of the type in which one or more liquids flowing over the probe produce at the probe a bound analyte complex that has a constituent that is capable of emitting light for detection by an external detector after the light passes from the analyte complex through a window of the cassette, wherein, after formation of the analyte complex within the cassette, the method of conducting the assay includes: (a) by liquid flow, removing liquid resident at the analyte complex on the enclosed surface and (b) forcing a stream of drying gas, such as air, to flow into the cassette, over the enclosed surface and out of the cassette during a drying interval under conditions that substantially volatilize and remove residue of the liquid associated with the analyte complex, and performing the detection on the dried complex enclosed within the cassette. (In such broad contexts, herein, the light-emitting constituent may be one or more tags associated with the bound material or some other constituent of the bound material. While the drying gas stream may be a sustained, constant gas stream, which is effective and efficient, the forced drying gas stream can also be varied in flow rate or interrupted once or a number of times, without detrimental effect except for related delay.)

Implementations of this aspect of invention may have one or more of the following features:

The method is conducted in a manner to form and dry an array of the analyte complexes at a corresponding array of surface-bound probes enclosed within the cassette, and includes performing the detection on the array of complexes within the cassette. Preferably in some cases, at least some of the analyte complexes in the array are on probes distributed transversely to the direction of liquid flow upon a width-wise extended surface, the passage is correspondingly wide, constructed to produce a uniform liquid flow over the array, and the drying gas is introduced through a relatively narrow channel and spreads to flow through the liquid passage over the array. In some implementations, the extended surface is a planar surface carrying a two dimensional array of probes and corresponding analyte complexes exposed to the liquid and drying gas flows. Preferably, in other cases, the probes of an array are arranged in a sequence in a line in a relatively narrow flow channel, the channel defining the reaction chamber.

The method is conducted in which an analyte complex comprises a biological receptor probe and biological ligands carried to the probe by liquid flow, for example the probe comprises oligonucleotide, peptide, polypeptide, protein or antibody, and the respective analyte comprises biomolecules that bind to the respective probe.

The method produces a complex which includes a light-emissive tag associated with the analyte complex. In some implementations of this feature, an analyte complex includes a fluorescent tag, for instance, the tag is a photo-excitable fluorescent compound that is stimulated to fluoresce by light from an external light source passing through a light-transmissive portion of the cassette. In some cases the stimulating light and the light for detection pass through the same light-transmissive window of the cassette. In other implementations, the tag is subject to electro-stimulation, an electrical pathway is provided in the cassette to the surface to which the complex is attached and the complex is stimulated by electro-stimulation via that pathway.

The method is conducted with steps (a) and (b) under automated control.

The method includes forcing the drying gas stream to flow over the enclosed surface for an interval of about one half minute or more.

The method includes forcing the drying gas stream to flow over complexes on a sequence of probes in a line in a relatively narrow flow channel.

The method employs heat delivered to the complex to promote volatilization of the liquid residue. In some implementations the heat is delivered to the complex at least in part by heating the drying gas before if flows into the cassette to a temperature above ambient but below a degradation temperature of the analyte complex or any associated tag. In some implementations, the heat is delivered to the complex at least in part by heating the surface to which the complex is bound to a temperature above ambient but below a degradation temperature of the analyte complex or any associated tag. In some of these cases an external heater is employed to heat the surface by thermal conduction from the exterior through a body portion of the cassette.

The method is conducted with the surface with bound complex being a microscope slide or segment of a microscope slide incorporated within the cassette.

The method is conducted with drying gas flow into or out of the cassette flowing through a device that prevents escape of liquid from the cassette.

During a wash phase prior to the drying gas flow, gas is caused to flow through a gas inlet channel to purge the channel of liquid contaminant. In a preferred case, bursts of such gas are introduced at spaced intervals during the wash phase.

According to specific aspects of invention, assays employ arrays of surface-attached binding agents or probes to which target biopolymer molecules in liquid solution bind. Methods are used to measure the concentration of analytes in the liquid fraction of the sample with a greater sensitivity and specificity than previously possible within an enclosed device. The assay development is carried out within a wide reaction chamber incorporating numerous separate capture regions addressed simultaneously, or within a flow channel where a linear array of capture regions encounter the liquid sequentially. Prior to inspecting the array for binding interaction, first, liquid is removed and replaced by air or other gas using liquid flow from the Reaction Chamber and from surfaces associated with detection (the array support as well as the window through which optical detection is conducted) and, second, these same surfaces are desiccated. Desiccation is performed by blowing a gas stream through the Reaction Chamber for a drying interval. The gas is air or nitrogen or any other non-reactive gas able to remove liquid and vapors of liquid from within the Reaction Chamber. The gas may be heated to promote desiccation. In addition or alternatively, the Reaction Chamber may also be heated. Temperatures employed may be 37 deg. C. or higher so long as the integrity of the complexes are preserved, in some cases as high as 75 deg. C., indeed up to about 90 deg. C. for biological complexes.

According to another aspect of invention, an assay cassette is provided which encloses a surface to which at least one probe is attached, the surface associated with a liquid passage that enables liquid flow over the probe, the cassette of the type enabling one or more liquids to flow over the probe to produce at the probe an analyte complex that has a constituent that is capable of emitting light that can pass through a window of the cassette for detection by an external detector, wherein, for use after formation of the analyte complex within the cassette, the cassette is constructed and arranged: (a) to enable, by liquid flow, removal of liquid resident at the analyte complex on the enclosed surface and (b) to enable a stream of drying gas to be forced to flow into the cassette, over the enclosed surface and out of the cassette during a drying interval under conditions that substantially volatilize and remove residue of the liquid associated with the analyte complex, to provide a dried complex within the cassette.

Implementations of this aspect of invention may have one or more of the following features.

The cassette has an array of surface-attached probes enclosed within the cassette, the cassette constructed to enable formation and drying of an array of analyte complexes corresponding to the array of surface-attached probes and constructed to enable detection of the array of complexes within the cassette. Preferably in some cases, the cassette has at least some of the probes distributed transversely to the direction of liquid flow, upon a width-wise extended surface, the liquid passage is correspondingly wide, constructed to produce a uniform liquid flow over the array, and the cassette is constructed to introduce the drying gas through a relatively narrow channel and to spread to flow through the liquid passage and over the array. In some implementations, the extended surface is a planar surface carrying a two dimensional array of probes exposed to the liquid and drying gas flows. Preferably, in other cases the probes are arranged as a sequence in a line in a relatively narrow flow channel.

In the cassette a probe comprises a biological receptor probe for biological ligands carried to the probe by liquid flow, for example the probe comprises oligonucleotide, peptide, polypeptide, protein or antibody.

The cassette contains a light-emissive tag material available to be associated with an analyte complex being formed in the cassette. In some implementations, the tag material is a fluorescent tag material, for instance, the tag material is a photo-excitable fluorescent compound, and the cassette includes a light-transmissive portion for transmitting stimulating light from an external light source to an analyte complex that includes the tag material. In some cases, the cassette is constructed to enable the stimulating light and the light for detection to pass through the same light-transmissive window of the cassette. In other implementations the tag material is subject to electro-stimulation, and an electrical pathway is provided in the cassette to the surface to which the probe is attached to enable a complex at the probe to be stimulated by electro-stimulation via that pathway.

The cassette is constructed to enable (a) and (b) to be performed under automated control.

The cassette is combined with a device adapted to force the drying gas stream to flow over the enclosed surface for an interval of about one half minute or more.

The cassette is combined with a device adapted to force the drying gas stream to flow over a sequence of probes in a line in a relatively narrow flow channel.

The cassette is constructed to enable heat to be delivered to the complex to promote volatilization of the liquid residue. In some implementations, the cassette is combined with a device to provide drying gas that has been heated before it flows into the cassette to a temperature above ambient but below degradation temperature of the analyte complex or any associated tag. In some implementations, the cassette is constructed to enable heat to be delivered to the complex at last in part by heating the surface to which the complex is bound to a temperature above ambient but below degradation temperature of the analyte complex or any associated tag. In some of these cases, the cassette is adapted for use with an external heater to heat the surface by thermal conduction from the exterior through a body portion of the cassette.

In the cassette, the surface to which the probe is bound is a microscope slide or segment of a microscope slide incorporated within the cassette.

In the cassette, drying gas flow into or out of the cassette is arranged to flow through a device that prevents escape of liquid from the cassette.

The cassette is combined with a control system constructed to produce gas flow during a wash phase for removal of liquid contaminants. In a preferred case the system is constructed to produce bursts of the gas at intervals during the wash phase.

The assay cassette has a common passage for introducing liquid and drying gas flows over the probe-bearing surface, the cassette having a bubble removal system to which at least some of the liquids are exposed before reaching the common passage, there being multiple connections to the common passage substantially upstream of the surface with attached probe but downstream of the bubble removal system, the connections including an inlet for liquid flow from the bubble removal system and another inlet arranged to receive the drying gas stream. In preferred implementations, there is a widening transition section between the common passage and the probe-bearing surface; the bubble removal system comprises a buoyancy chamber through which liquids flow; all of the liquids of the assay are forced to flow through a buoyancy chamber; the cassette has storage volumes on board the cassette for all liquids employed in the assay, and the assay cassette is combined with an air pump for producing the stream of drying air and a liquid pumping system for the liquids of the assay, preferably the liquid pumping system being a liquid diaphragm pumping system.

According to specific aspects of invention, the cassette incorporates one or more fluidic components such as compartments, wells, chambers, traps, bubble traps, fluidic conduits, fluid ports or vents, gas intake arrangements, valves, and the like. In case of complexes subject to photoexcitation, an excitation pathway is also provided (e.g. by one or more light-transmissive components configured to enable passage of light into the cassette to produce excitation of a light-emitting constituent of an analyte complex). The cassette is associated with one or more detection components or sensors and detection windows (e.g. windows configured to allow optical measurements on samples in the cassette such as fluorescence, phosphorescence, chemiluminescence, electrochemiluminescence and the like). A cassette may also store reagents for carrying out an assay such as binding reagents, detectable labels, sample processing reagents, wash solutions, buffers, etc. The reagents may be present in liquid form, solid form and/or immobilized on the surface of solid phase supports present in the cassette. In certain implementations the cassette includes all the components necessary for carrying out an assay. In some implementations, a cassette reader is included which is adapted to receive the cassette and carry out certain operations on the cassette such as controlling liquid and gas movement, heating the gas, supplying power, conducting physical measurements on the cartridge, and the like.

In preferred cases, the methods and cassettes described here also have one or more of the following features:

The surface enclosed within the cassette supports immobilized binding domains of protein.

The surface enclosed within the cassette supports immobilized binding domains of genomic nature, such as oligonucleotides, SNPs, segments of genes, etc.

The surface enclosed within the cassette supports immobilized binding domains of cells or cell lysate.

The surface enclosed within the cassette supports immobilized binding peptides.

The surface enclosed within the cassette supports immobilized binding ligands comprising small organic molecules having molecular weight between about 500 to 6000 Daltons (including steroids, peptides and expression regulators such as RNA primers and siRNA).

According to another aspect of invention, an external reader station for an assay cassette is provided which includes a system for causing liquid flows that cause formation of a light-emitting complexes on a surface enclosed by the cassette and a detector for detecting light emitted from the complex that passes through a window of the cassette, the reader station including a source of pressurized drying gas and a control constructed to automatically produce a sustained stream of drying gas into and through the cassette, to dry the complex before light detection.

Implementations of this aspect of invention may have one or more of the following features.

The external station includes a heater for providing heat to the complex to promote drying. In some preferred cases the heater is arranged to heat the drying gas before it enters the cassette. In some preferred cases a heater is arranged to heat a surface of the cassette that is in heat-transfer relation to the complex or to drying gas flowing to the complex. In some cases, for drying, a surface of the cassette is heated to temperature above that employed during formation of the complex.

The external reader station includes an air pump for producing the stream of drying air and a liquid pumping system for the liquids of the assay, in preferred implementations the liquid pumping system comprising a linear actuator constructed and arranged to deflect a liquid pumping diaphragm of the cassette.

The observed effectiveness of the drying air stream through the enclosed cassette may be attributable to more than one physical light-emitting phenomenon described in scientific literature. For instance, inhibition of quenching and nonradiative energy transfer from electronically excited tag molecules via molecules of liquid solution may occur due to removing traces of residual liquid. Change of physical structure of tag molecules in manner enhancing their light-emitting capability may occur due to removing residual traces of liquid.

### BRIEF DESCRIPTION OF THE DRAWINGS

FIG. 1 shows, by isometric diagram, a wide capture surface in a reaction chamber exposed to a flow of analyte and other reagent.

FIG. 2 is an isometric view of a flow system within a cassette employing a reactive chamber as shown in FIG. 1.

FIG. 2a is a diagram of an external system that produces a drying air stream through a cassette.

FIG. 3 is a diagram of a generalized disposable assay cassette and steps performed on the cassette.

FIG. 4 is a more detailed diagram of one general implementation of a disposable cassette with activatable components to perform an immunoassay of sandwich type.

FIGS. 4A, B and C are front, back and perspective views, respectively, of the molded central body of a particular implementation of a cassette of the type of FIG. 4.

FIGS. 5A and 5B show a one way “duck bill” gas flow valve that enables flow of gas in a cassette.

FIG. 6 is a schematic illustration of a passive microfluidic capillary burst valve useful in the gas flow path.

FIG. 7 illustrates the reading of the results of the assay when photostimulated fluorescent tags are employed.

FIGS. 8 and 8A are graphs showing the evolution of the intensity of the fluorescence emission as desiccation progresses.

FIGS. 9 and 9A are graphs showing the advancement of the assay sensitivity as the desiccation progresses.

FIG. 10A is a graph showing the rise of the signal-less-background of an assay as the drying air stream flows through the reaction chamber. FIG. 10B shows deterioration to the original state as the reaction chamber is rehydrated.

FIG. 11 is a cross section of an air heater useful to heat air before entering the cassette.

FIG. 11A shows an external heater at a reader station for heating the cassette.

FIG. 12 shows an air inlet feature of a cassette that employs a gas-transmissive membrane while FIG. 12A is a plan view of that feature.

FIGS. 13 and 13A show the membrane element.

FIG. 14 is an exploded diagram of a closed cassette employing a narrow channel as the reaction chamber.

FIG. 15 is a diagram of a closed cassette constructed for electro-stimulation of fluorescence.

### DETAILED DESCRIPTION OF PREFERRED IMPLEMENTATIONS

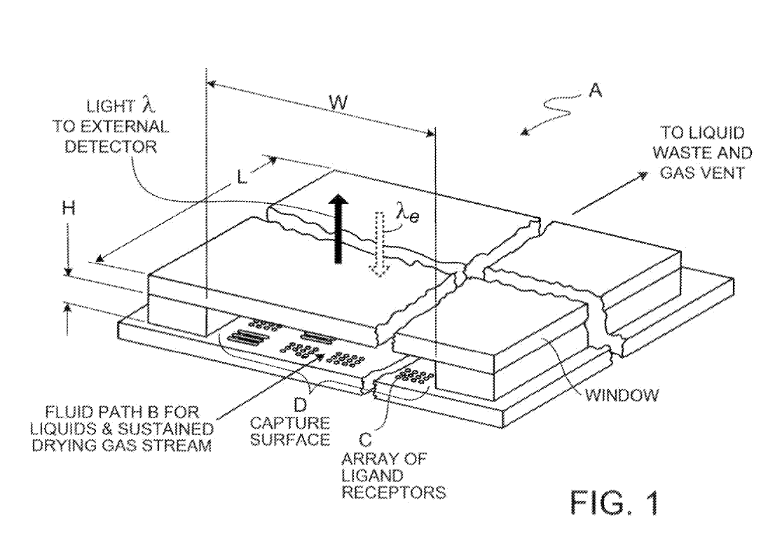

FIG. 1 shows cassette reaction chamber A receiving flow along flow path B. A ligand-containing liquid flows over two-dimensional array C of ligand receptors (capture reagent or probe) on a capture surface D. In preferred implementations, characteristic dimensions of the two-dimensional array C are greater than about 0.1 cm; in one preferred implementation the capture surface F is 4 mm wide and 12.5 mm long. The gap height H which determines the depth of sheet-form flow over the capture surface is preferably between about 30 and 300 μm (micron). In some preferred cases, gap H is between 50 and 150 μm. The array C of capture reagent is located at a safe distance from the channel corners, typically 0.5 mm. This helps avoid any gas micro-bubbles that might form upon liquid heating and grow in the corners. The array may comprise a pattern of spots of capture elements (probes) that form an assay. Spots may comprise nucleic acid segments, oligonucleotide segments, proteins, peptides, cells, cell lysates or other probes normally used to detect or identify biological effect. In other cases the probes may be synthesized genomic probes or the like. Liquid flow along path B may be the analyte, associated reagents or light-emissive tags associated with an assay.

After analyte complexes are formed at the probes by liquid flowing over the array and complexes have been labeled with fluorescent tags, liquid is removed by liquid flow and replaced by gas, e.g. air or an inert gas.

According to inventive aspects discussed, a drying (desiccating) gas stream such as air is then propelled by a source through the reaction chamber to dry the capture surface along with analyte complexes, i.e. to volatilize and remove liquid residue at the surfaces and the opposed window as well.

In some instances an initial flow of gas from the same gas source is used to force the liquid to flow from the chamber, preceding the sustained drying gas stream.

After the drying interval, light λ from the complexes is emitted and passes through the window to an external detector. In the case of use of photo-excitable tags, excitation light λ_{c} may be directed from the outside to excite the emission of light λ from the complexes.

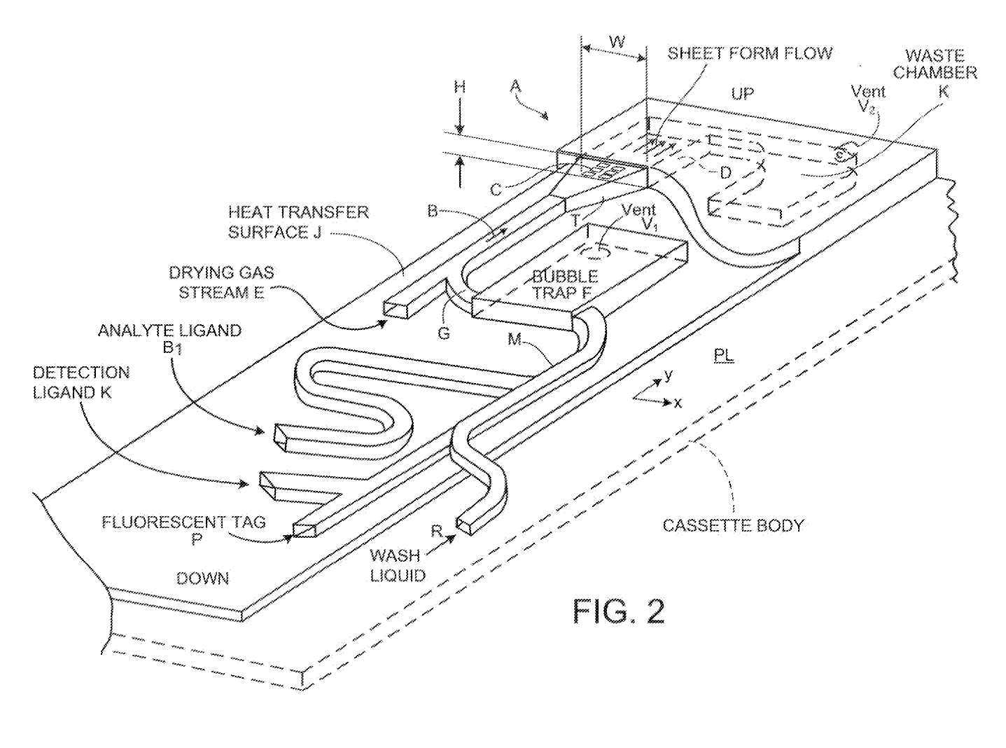

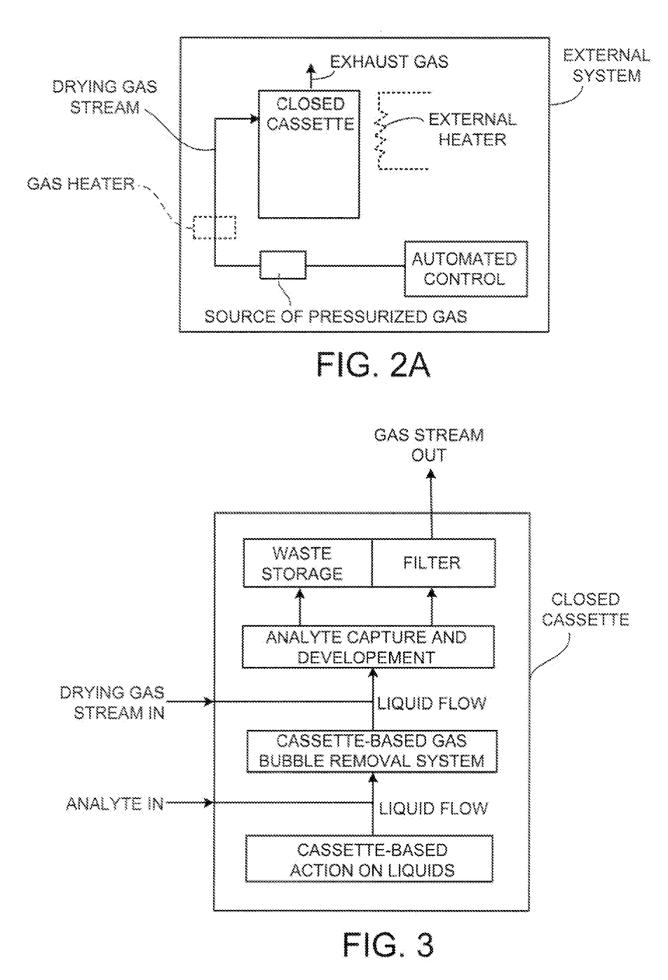

FIG. 2 is a three dimensional representation of the functions of an assay as they operate with respect to a reaction chamber A such as shown on FIG. 1. The reaction chamber is provided in a cassette having an on-board bubble removal system for the liquids. In the implementation shown it is a bubble trap F in the form of a buoyancy chamber.

Analyte ligand B₁, detection ligand K, fluorescent tag P and wash reagent R flow through bubble trap F, then through channel B and the reaction chamber A in proper sequence relevant to the performance of an assay. A sustained drying (desiccating) stream E of gas, e.g. air, is then directed into common channel B at a “Y” connector located downstream from bubble trap F, to enable flow of the gas through channel B to the reaction chamber A. The gas performs an initial function of displacing liquid within the reaction chamber; then, as a sustained flow, the drying gas stream volatilizes and removes residue liquid such as adsorbed liquid and liquid vapor from within the reaction chamber A. A transparent window shown in FIG. 1, not shown in FIG. 2, is preferably of glass or plastic having minimum fluorescence capacity or having uniform fluorescence at the wavelengths associated with the fluorescence of the probe. The window may be made from micro cover glass, VWR part #48404-452 or made from Polycarbonate Film from Tekra/Bayer and may be coated with titanium dioxide (TiO₂₎ or silane (SiH₄) or subjected to other processes intended to enhance fluid flow.

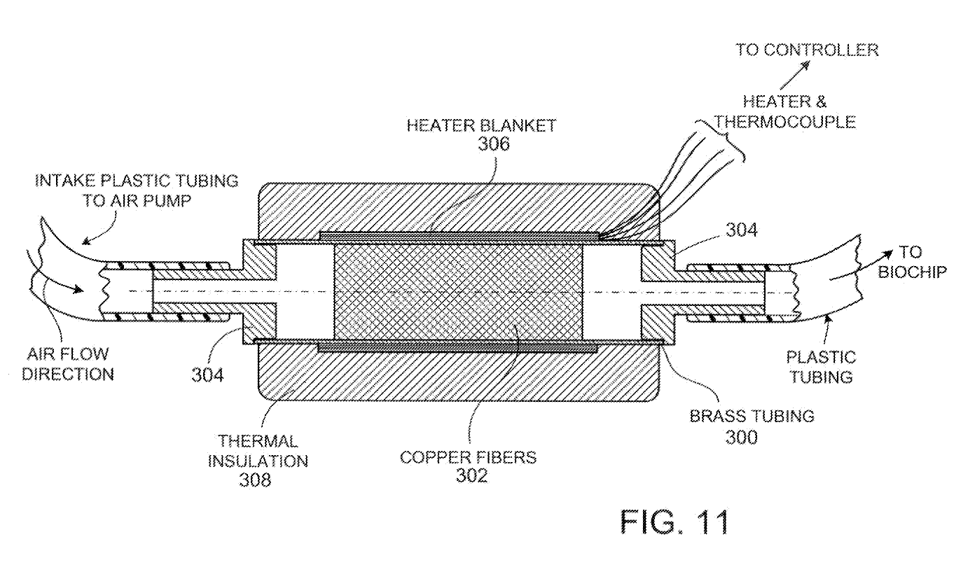

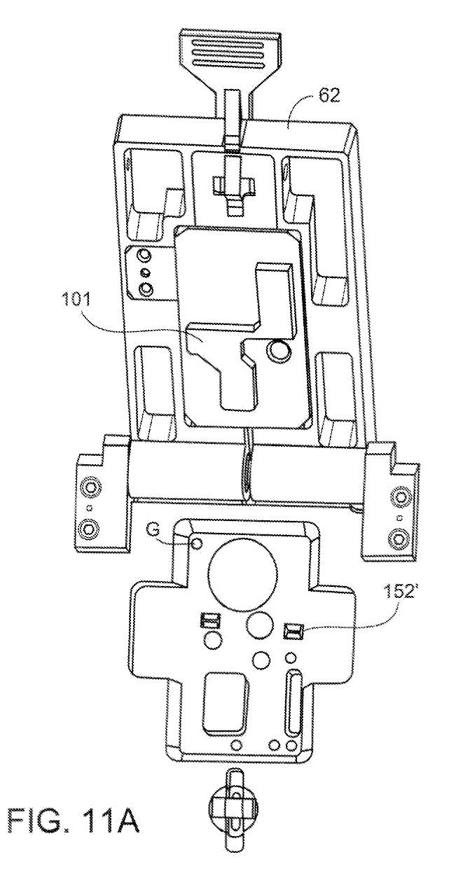

Each liquid as well as the drying gas can be brought to approximately assay temperature as it flows over the heat transfer surface of the cassette prior to entering the reaction chamber. Heat may be conducted to the heat transfer surface J through a wall of the cassette from an external heater, see FIGS. 11A. Additional heat may be added to the drying gas before it enters the cassette.

Successive, continuous, timed liquid flows of analyte ligand, detection ligand, fluorescent tag, and washing liquid are produced by forces applied to the liquids upstream of bubble trap F under automated control of an operating system. They enter common passage B through one inlet branch of the “Y” connector, and proceed through reaction chamber A.

The gas is then caused by the operating system to enter common passage B through the other inlet branch of the “Y” connector. A gas flow of one or a few seconds displaces the liquid from Chamber A. The operating system then maintains a sustained drying gas stream e.g., of ½ to 2 minute duration for drying (desiccating) the chamber and capture surface. The flow passage for path B following the “Y” connector is narrow relative to the width of the reaction chamber. The flows of liquids and gas broaden in a transition region T to the full width of the chamber. The chamber surface carries a two dimensional array C of probes

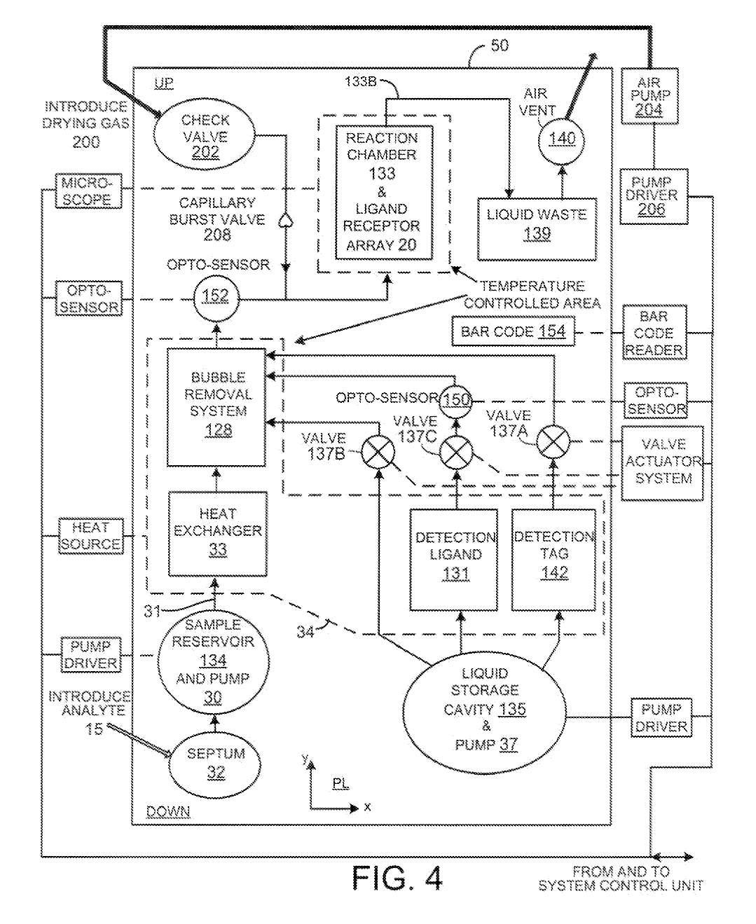

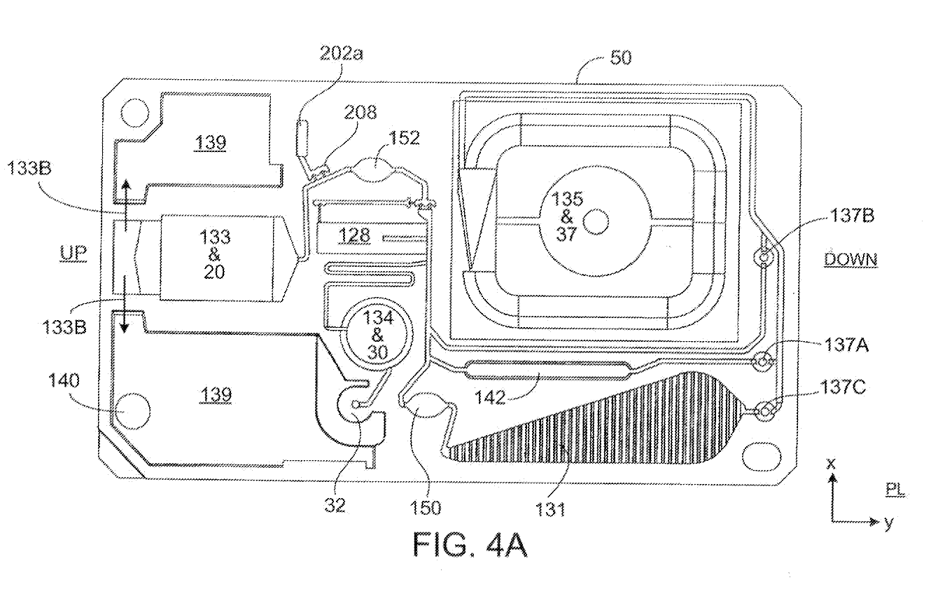

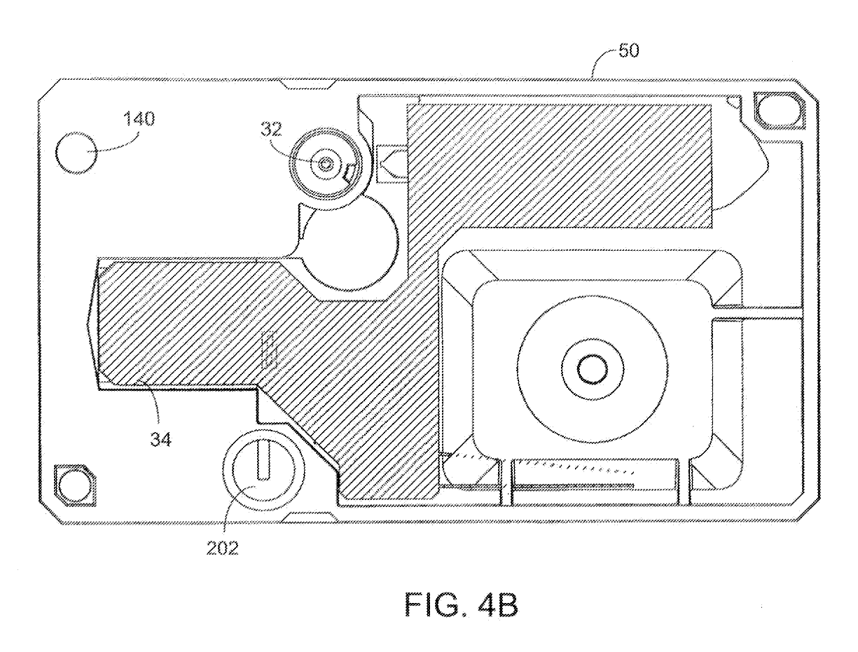

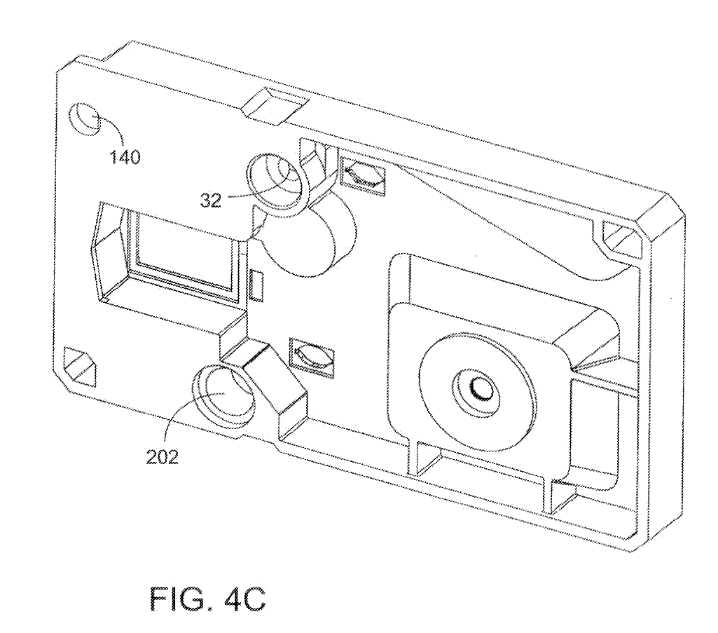

A further aspect of invention for the gas-dried system of FIG. 2 is the possibility, owing to the slow flow characteristics of the preferred system, that all liquids may be stored in suitable quantity on-board the cassette, as suggested in FIG. 3, while the gas stream required for drying of the reaction chamber is introduced from the exterior. FIGS. 4-4C show particular implementations of this arrangement. According to the general principle of FIG. 2, however, one or more of the liquids for the assay may also originate from exterior of the cassette.

Another feature illustrated in FIG. 2 is a waste chamber system K that is sized to receive and contain all waste liquids from the assay. It is vented at V₂ to enable flow of displaced air and drying gas of the interior of the cassette.

The components are shown provided in a generally planar format in FIG. 2. In operation the plane is inclined to the horizontal with the reaction chamber A highest. Besides orienting the bubble trap buoyancy chamber above the liquid flow path origin, the inclination enables uniform upward sheet-form liquid flow over the array C on the capture surface, and thence, by gravity flow, to the waste system K.

An air vent arrangement V₁, at the buoyancy chamber, enables the buoyancy chamber to be filled with liquid during initiation of the assay. The air vent V₂ at the waste chamber enables air to be expelled from the liquid passage system during initiating phases of the assay and during gas flow that flushes out the liquid, as well as enabling the drying gas stream for the interval that desiccates the analyte complexes on the walls of the reaction chamber. The vents do not obstruct the passage of the gas but prevent the passage of liquid and of contaminating liquid-borne substances such as pathogens. Thus after the performance of the assay, the planar cassette may be placed horizontally without escape of the liquid. FIG. 2A illustrates the external system associated with a cassette. It includes a source of pressurized gas. Optional heating is provided by an optional external heater acting upon the cassette and an optional gas heater which heats the drying gas before the gas enters the closed cassette. They may be employed individually or may be employed together to enhance performance. Not shown is the separate liquid pumping arrangement that propels the liquids.

FIG. 3 illustrates an assay cassette having all reagents as well as liquid waste stored on the cassette. Assay cassette systems employing the concept of FIG. 3 enable improved performance of complex assays with drying of the support within the closed cassette, without need for expert personnel.

Referring to FIG. 4, the concepts of FIGS. 1, 2 and 3 and other significant features are combined to provide a highly effective self-contained assay cassette. Though the flow features are useful in general for receptor-ligand assays, they are shown adapted for an immunoassay. The assay is identified by a bar code 154. FIGS. 4A, 4B and 4C show a molded plastic body for implementation of the cassette of the type of FIG. 4, like numerals indicating the location of like functions.

In FIGS. 4 and 4A, B and C, liquid sample 15 containing the analyte, e.g. blood serum, is introduced by needle or pipette through a septum 32 to a sample chamber or reservoir 134 within cassette 50. A displacement pump 30 (e.g., a diaphragm of the cassette operated by electrically driven linear actuator of the reader unit) is associated with sample reservoir 134 for forcing sample 15 through passage 31 into other regions of the system. Heat exchanger 33 heats liquid sample 15 while other parts of heating system 34 heat other liquids for the assay. The heating system may be arranged to bring the liquids to approximately assaying temperature, e.g. physiological temperature, 37° C. For other parts of a liquid containment system, liquid storage cavity or chamber 135 is provided, e.g. for buffer solution, which is associated with displacement pump 37 (which also may be a diaphragm of the cassette operated by a linear actuator of the reader unit). Pump 37 provides forced liquid flow as needed to support the assay, e.g. wash liquid and liquid to chambers 131 and 142 to liquefy, dilute or mix other reagents contained within the body of cassette 50. For an immunoassay, dry detection antibody may be coated on a surface of chamber 131 and dry fluorescent tag coated on a surface of chamber 142.

A bubble removal system 128 is shown generically. In FIG. 4 it is implemented as a gravity-based bubble trap or buoyancy chamber divided into two compartments shown in FIG. 4A. It receives a succession of continuous liquid flows from chambers 134, 131 and 142. It removes micro-bubbles produced previously in the liquids. Following the bubble trap the liquids proceed, in controlled succession, along a narrow common path, thence through a widening transition passage and through reaction chamber 133 for exposure to a capture surface carrying array 20 of ligand receptors. The liquid flows are produced by a system of passages controlled by externally driven liquid pumps 30 and 37 and externally activated valves 137A, B and C driven in response to sensors 150 and 152 that sense arrival of liquid by the arrival of a liquid-air interface in the respective passages. The sensors and the liquid pump and valve actuators as well as the electronic control system may all be at the reader station as described in the below referenced patent application which are hereby incorporated by reference in these respects.

Waste chamber 139 (provided as two chambers in the implementation of FIGS. 4A, B and C) receives liquid waste via passage 133B from flows through the reaction chamber 133. As indicated by “up” and “down”, the plane PL of the cassette is oriented at a substantial angle to horizontal during use to produce gravity flow from the reaction chamber 133 to waste chamber 139. The waste chamber is vented at 140 and the upward flow arrangement of the passage system to the waste outlet enables air in the system to be expelled by action of the displacement pumps 30 and 37 prior to initiation of the assay. Venting may be through conventioned Porex filters defining gas path lengths longer than about 2 mm, having pore size between about 15 and 50 micron.

Under control of the external system control unit, after completion of formation of analyte complexes with fluorescent tags attached at the array 20 of probes on the capture surface, air or gas 200 is introduced through check valve 202 (located at the back of the cassette, FIG 4B in the implementation) and through capillary burst valve 208. Gas may be supplied from an external source under pressure or from a small air pump 204 incorporated in the assay external equipment. A suitable pump is model NMP05 from KNF Neuberger, Inc. in Trenton, N.J. It may be used to produce a stream of preferably filtered air. In such a case, pump 204 may be energized by pump driver 206 connected to the system control unit. An air filter at the intake of the air pump may employ a “Whatman” filter from Whatman Plc, Brentford, Middlesex, UK, having thickness of about 200 micron and 0.45 micron pore size.

FIG. 4 also illustrates the functional relation between other features of the cassette and features of the external apparatus.

For further explanation of details of the construction and operation of the cassette and the heating and reading station that automate the assay, see U.S. Ser. No. 11/262,115, filed Oct. 27, 2005, and PCT/US2005/0390, filed Oct. 27, 2005, each entitled, “ASSAYS BASED ON LIQUID FLOW OVER ARRAYS”, each of which is herein incorporated by reference.

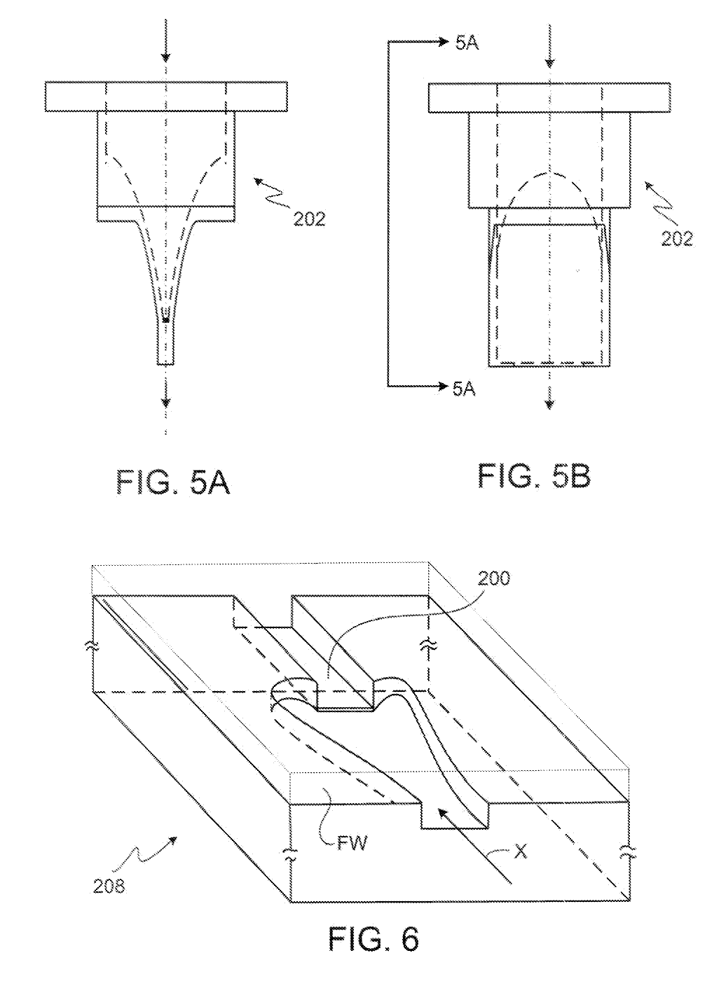

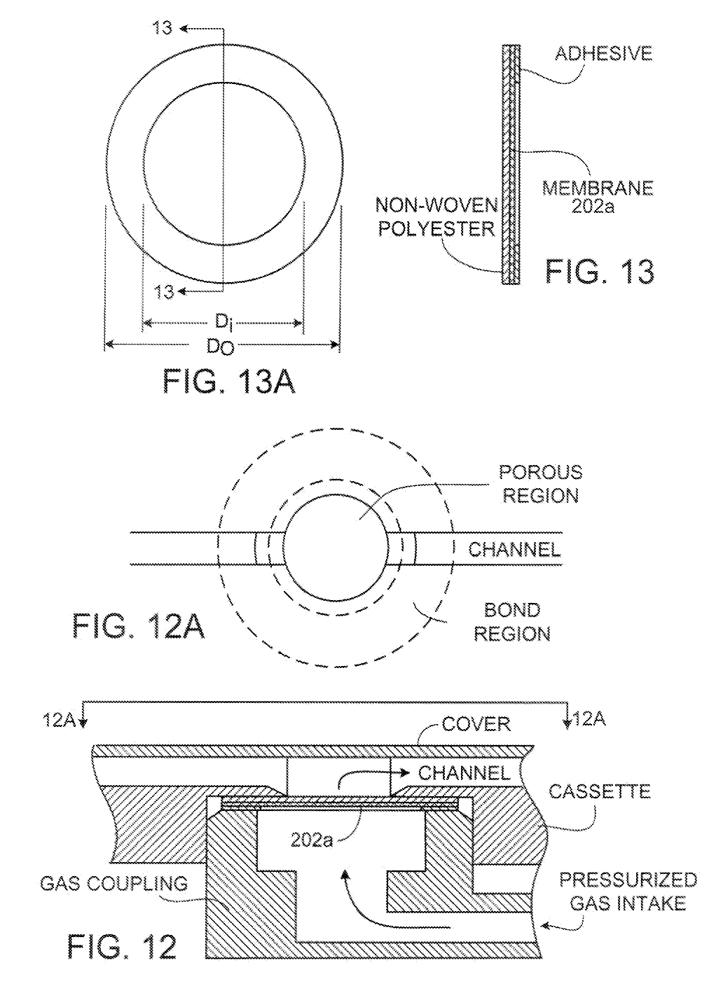

FIGS. 5A and 5B show a duckbill resilient check valve 202 for use as an entry port for gas into the cassette. Its mating resilient bill-like members permit gas flow outward between the members, in the direction of the arrow, while preventing liquid flow in the opposite direction. Such a valve may be obtained from Vernay Inc. in Yellow Springs, Ohio. See FIGS. 12-13A for an implementation which substitutes for check valve 202 a liquid-restrictive, gas-permeable membrane 202a.

FIG. 6 show the construction of capillary burst valve 208 is employed to prevent liquid from entering the check valve channel but permits unimpeded air flow in the direction of arrow X into the reaction chamber through channel 200. More than one burst valve may be used in parallel to minimize air flow pressure drop. Two are employed in the implementation of FIGS. 4A-4C, as indicated.

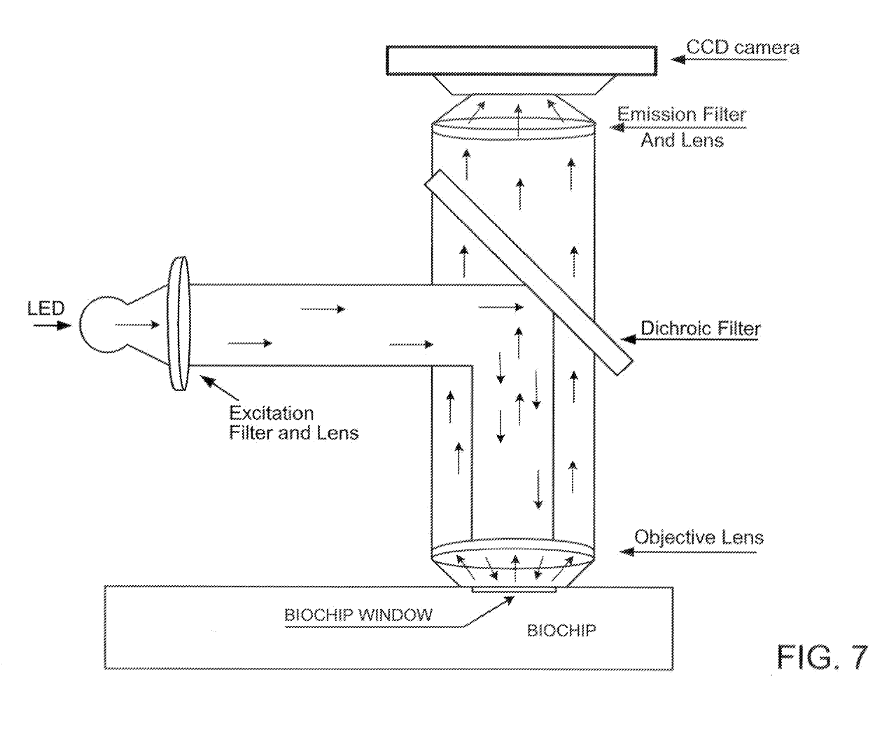

FIG. 7 is a symbolic presentation of a CCD camera based epi fluorescent microscope useful to read cassettes made according to FIGS. 1, 2, 3, and 4. The CCD camera is model ST-402XME from SBIG, Inc., in Santa Barbara, Calif. It is used to capture images of the fluorescent energy that passes from the analyte to complexes through the window of the cassette (biochip) after the sustained stream of drying air has flowed through the reaction chamber of the cassette.

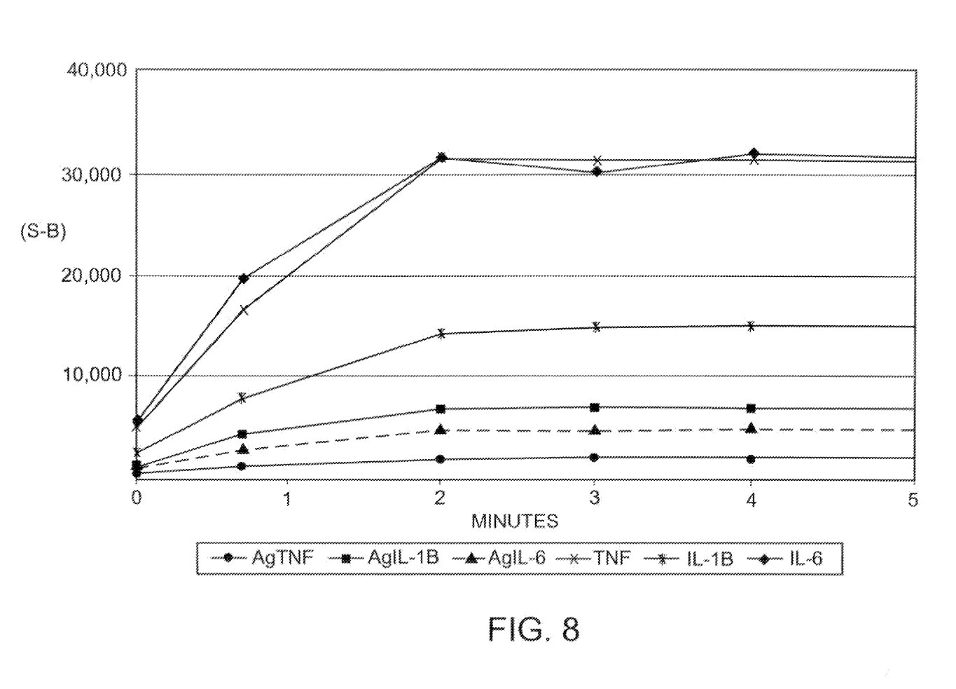

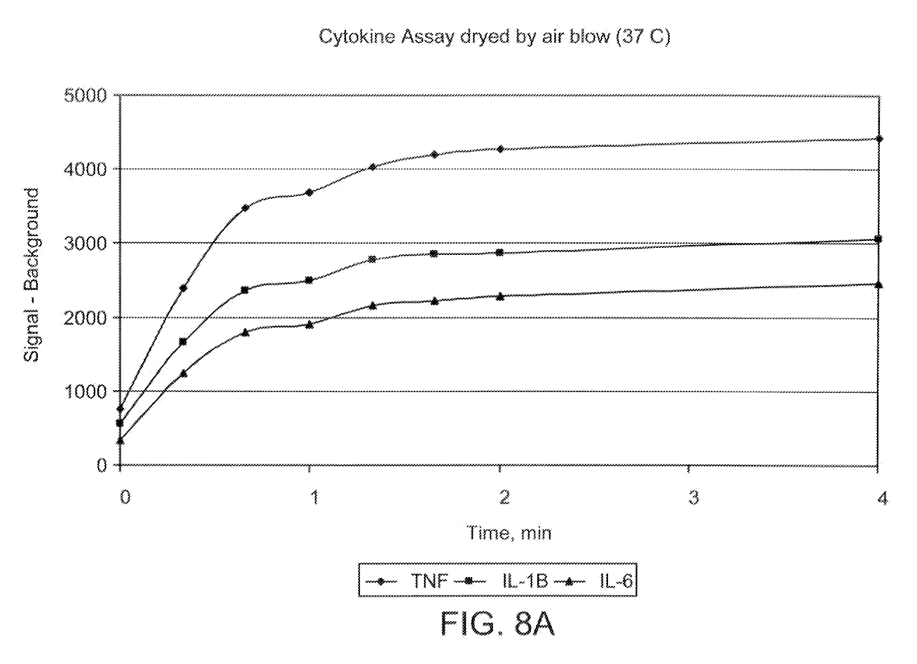

FIG. 8 shows the evolution of the fluorescence signals of a completed multiplexed cytokine assay performed on the cassette simulation mentioned above. Analytes TNF, IL-IB and IL-6 were employed along with corresponding controls AgTNF, AgIL-IB and AgIL-6, according to the legend. An arbitrary concentration of the analytes was employed. The fluorescent image of the array was captured on a CCD camera similar to that shown in FIG. 7. FIG. 8 shows data derived from images captured from the start of flowing air into the reaction chamber under the conditions outlined above. Data at time 0 was captured prior to any air flow. It can be seen that the signal less background level (S-B) increased rapidly as air flow continued. The background signal level, i.e., from areas of the surface not bearing a probe is subtracted as is normal because it originates from the digital or electronic signal processing and from the optical system, and hence is unrelated to the fluorescence emitted by the analyte complexes under study. (The scale of the plot depends upon the concentration of the analyte tested and the time of exposure at each measurement point.)

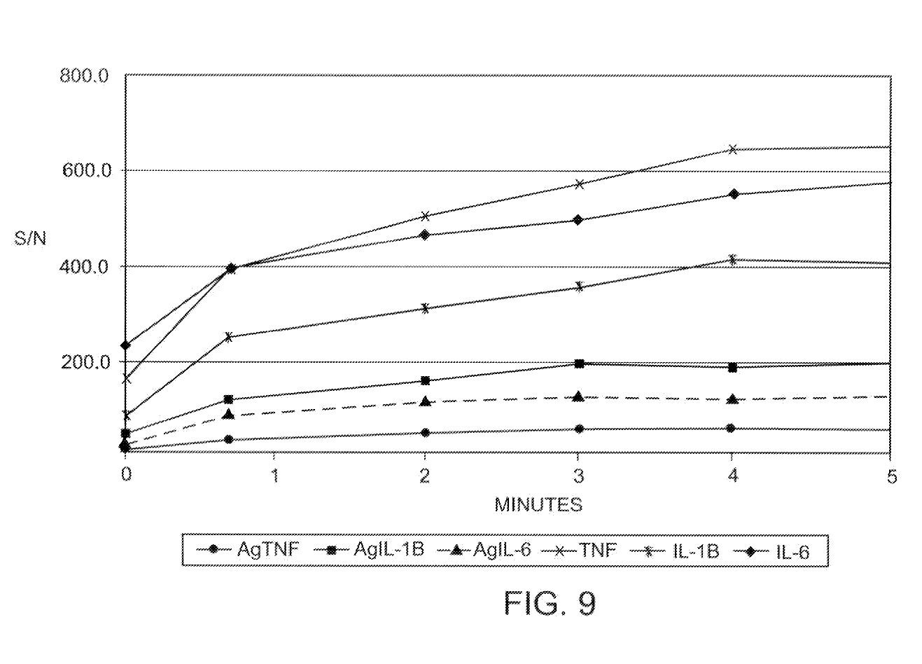

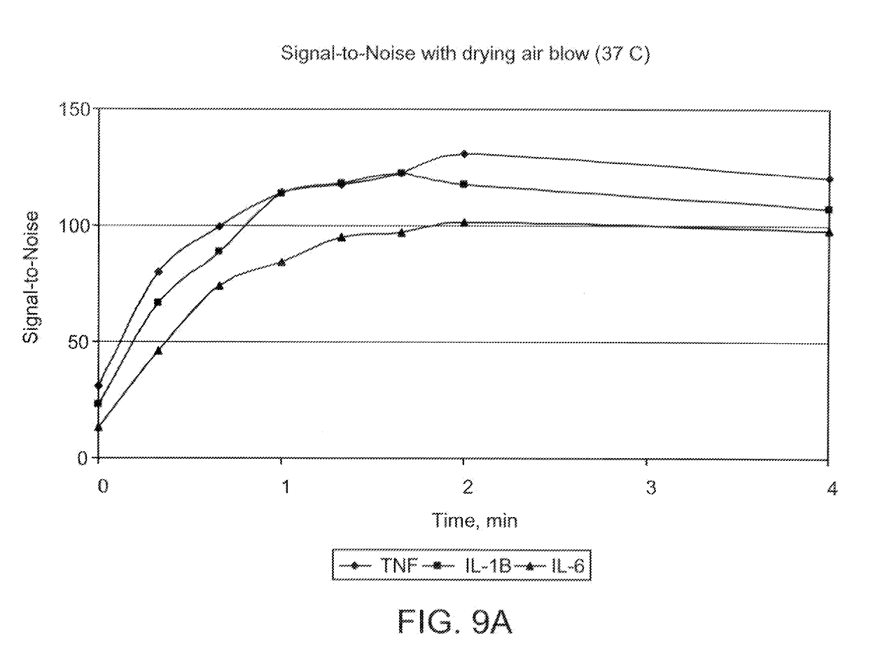

The sensitivity of the assay is defined by the signal-to-noise ratio (S/N) as shown on FIG. 9 where the noise is defined as the standard deviation of the signal received from the region surrounding a spot containing a capture antibody selected to represent a specific assay. A number of software packages may be used to perform this derivation. The software used in this derivation was Jaguar 2.0 from Affymetrix and ported to Excel for presentation. It can be seen that the major part of the improvement is signal to noise ratio occurred within the first minute of the sustained drying airflow and that about 80% of the improvement occurred within the first two minutes.

FIGS. 8A and 9A, are similar graphs, respectively, obtained using the fully implemented cassette described in respect of FIGS. 4A-4C. A lower concentration of the analyte was employed resulting in a different scale for the ordinate of the FIG. 8A graph. The temperature of the forced air stream was controlled to 37 C. Graphs 8A and 9A show even more rapid improvement of measurements due to the drying air stream.

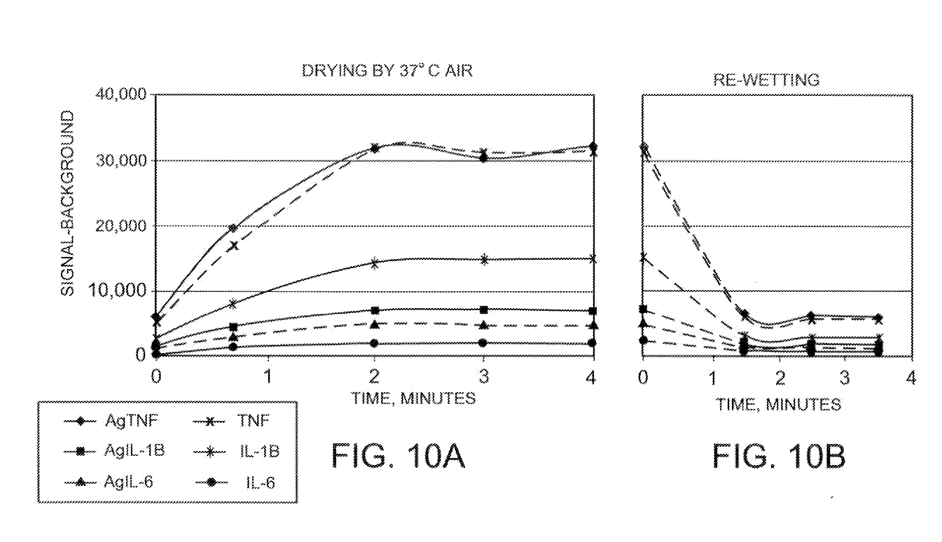

FIGS. 10A (corresponding to FIG. 8) and FIG. 10B are graphic presentations respectively of the meaningful fluorescent energy produced by an assay as a drying air stream is sustained through the reaction chamber (FIG. 10A) and after rewetting (FIG. 10B). The data was transferred to Excel for analysis and display. It should be noted that rewetting necessitated removal of the cassette from the instrument causing a delay and uncertainty in data acquisition indicated by the dotted line in the re-wetting graph.

Protocol for the Preferred Implementation

Using the cassette of FIGS. 4A-4C, following completion of the liquid phases of the assay that forms fluorescently labeled analyte complexes on the enclosed surface, liquid is removed from the reaction chamber. This may be achieved by draining, suction or blowing an initial flow of air (or other nonreactive gas) through the reaction chamber. This is important because reagent and/or other liquids contain salts and/or molecules that can remain on the wall of the reaction chamber as evaporation takes place. These desiccated remnants may also be fluorescent and add to the background signal. In an alternate manner, the reaction chamber may be washed with a liquid free of any such component.

The reaction chamber may then be heated to a temperature higher than 37 degrees C. in order to accelerate desiccation. Depending on the assay, temperature range from 50 Deg. C. to 90 Deg. C. may be employed. The upper limit of heating is dependent on the temperature at which the detected assay signal degrades, which may be attributable to a number of factors, governed by the materials employed in the assay. For any given assay, a trial series over a range of temperatures is employed to determine the maximum permissible temperature. Temperature of the reaction chamber can be regulated with a heater or by a flow of suitably heated air.

In the preferred implementation, a reaction chamber with a section of approximately 4.1×12.5×0.1 mm, width, length and height, respectively, is subjected to a pressure differential of approximately 100 mbar and an air flow of 200 cc/min across its length. Liquid is quickly removed by liquid flow and greater than 90% of optimum signal, S, and signal-to-noise ratio, S/N ratio, is obtainable in less than 4 minutes with the air temperature held at 37 Deg. C., see FIGS. 8-10A.

Desiccation can be accelerated if the air is heated or the reaction chamber temperature is raised following liquid removal.

As an added feature, to deal with the possibility that contaminant liquids such as analyte or detection (tag) molecules may have lodged in the air inlet channel during conduct of the assay, it is found useful to blow back the liquid during the wash phase of the liquid process. This is achievable by blowing air at the start of the wash cycle (wash phase) to force the materials into the wash flow. This may be accomplished by blowing air or gas through the inlet for 2 seconds at a time 1 minute after the start of the wash cycle. This may also be repeated a number of times at spaced intervals, for instance, 2 to 5 times during progress of the wash cycle, preferably 3 to 4 times. The gas pulses tend to agitate the liquid and dislodge any contaminant, and are of sufficiently short duration to avoid premature desiccation of the complexes. They have benefit in assuring effective wash and removal of unbound, noise-producing fluorescent tag material as an independent feature.

Additional Features

FIG. 11 shows a suitable compact air heater. A 45 mm length of brass tubing 300 with a wall thickness about 0.35 mm is stuffed with about 4.5 g. of copper fibers 302 such as obtained from a copper scrubber—Chorboy distributed by Reckitt Benckisser Inc. Parsipany N.J. The copper fiber slug is compressed to approximately 13% of the density of copper and centered. Both free ends of the brass tubing 300 are fitted with commercial plastic pipe fittings 304 such as part B-TCNY-A1 from Small Part Inc. of Miami Lakes, Fla. A 50 ohm heater blanket 306 from Minco Inc. of Minneapolis, Minn. is wrapped around the exterior of the brass tubing 300 and approximately centered. A 20 mm length of insulating sleeve 308 preferably such as used to insulate ½ inch hot water house piping is slid over the assembly to minimize energy loss. A thermocouple is inserted to sense and enable control of the assembly temperature. Air flowing through the tube is heated by flowing over the heat conductive surfaces provided by the tubing and the copper fiber filling. An alternate design may use copper or aluminum foam with, for instance, a 15% metal density instead of the copper fiber material. Other arrangements are of course feasible.

The cassette of FIGS. 4A-4c is constructed to be heated from its back side (side opposite from its window), see FIG. 4B. The shaded region represents the presence of an external heater 101 in a receiving cavity in the backside of the cassette. Referring to FIG. 11A, a cover at the external reader station carries heater 101. After insertion of the cassette into a receptacle of the station, the cover is closed, bringing heater 101 into heat transfer relation to the back of the cassette. Heat passes through the wall of the cassette, effectively heating the region 34 of FIG. 4. This heats the reaction chamber 133 to enhance the volatilization of liquid residue during passage of the drying gas stream through the cassette. The heating of the cassette surface can raise the temperature of the drying gas before it reaches the complex, thus heating the complex by convection, and can heat the surface on which the complex resides and thereby the complex, by conduction. For further details concerning heater 101 see the above-cited patent applications, which in this respect are also incorporated by reference.

In FIGS. 12-13A is shown a membrane vent available from W. R. Gore, VE 40308, and its method of installation. It is shown as a substitute for the duck bill valve of FIGS. 5A and 5B. The central portion of the vent is permeable to gas but impermeable to water. As shown, pressurized gas, under control of the external unit, applies pressurized gas at the intake, which sustains a drying gas flow as described, see arrows.

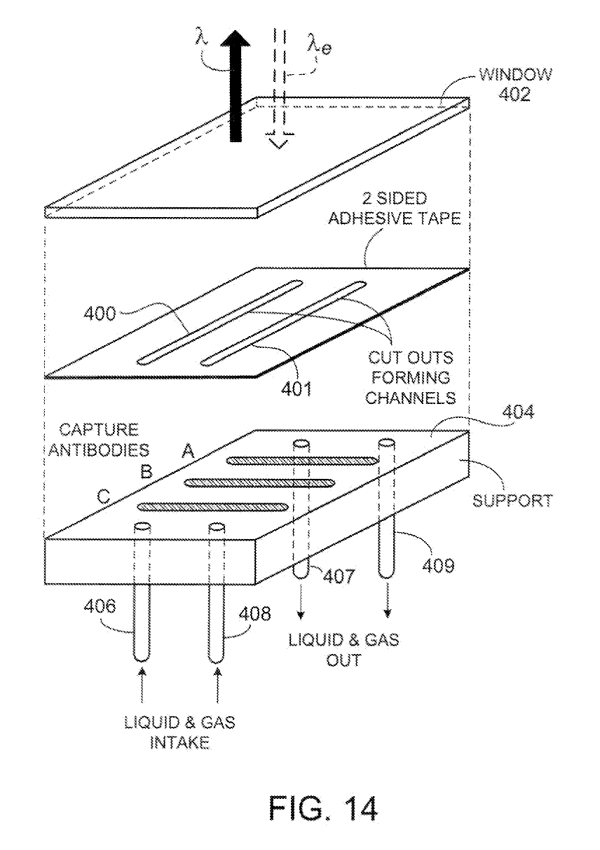

FIG. 14 is an exploded diagrammatic view of a reaction chamber of a cassette that defines two narrow flow channels 400 and 401 covered by a light-transmissive window 402. Each channel may have a flow cross section of 100 micron width and 35 micron depth. An array comprising a sequence of surface-attached probes A, B and C (attached to closing surface 404) are arranged in sequence along the length of the channel for exposure sequentially to flow from respective inlet conduits 406 and 408. After flowing through the channels, the flows discharge through respective outlet conduits 407 and 409. As in the previously described implementations, analyte, reagent, tag and wash liquids flow through the channels to develop an array of labeled analyte complexes distributed along the channels. After formation of the labeled complexes, following washing, a sustained stream of drying gas is forced to flow into the cassette through inlet conduits 406 and 408, through the channel 400 and 40 and out through conduits 407 and 409, respectively. Subsequently, conditions are provided to cause the tags to emit light λ which passes through the window to an external detector. Excitation may be by light passing through the window λ_{c} into the chamber or may be by an externally controlled electrical voltage applied through electrical conductors.

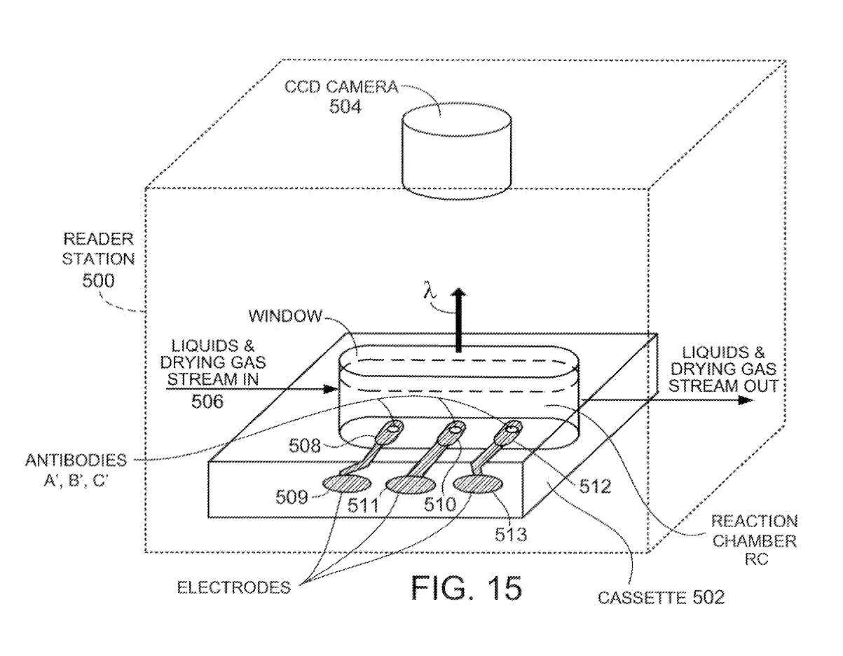

FIG. 15 represents diagrammatically a reader station 500 constructed to receive a cassette designed for electro-simulation of photo-emission. The reader station has circuitry not shown, for introducing electro-stimulation to the cassette. It has a CCD camera 504 arranged to form an image of the light pattern that passes through the cassette window 505 from the electro-stimulated analyte complexes within the cassette.

To form those complexes, an array of capture antibodies is attached to corresponding electrically conductive members on a surface of a reaction chamber. As with the previous implementations, the assay liquids and the drying gas stream flow through the reaction chamber.

The reaction chamber and array of conductive surfaces may be of various forms. For illustration, the reactive chamber RC in FIG. 15 is shown as a linear channel similar to that of FIG. 14 and conductive members or pads 508, 510, and 512 are shown in sequence along the length of the flow channel. Antibodies A′, B′ and C′ are attached respectively to the conductive members. Each pad is associated with an electrode 509, 511 and 513 respectively, for receiving electrical excitation from the reader station to stimulate photo-emissions from the respective analyte to complexes. Other conductors, not shown, may be employed to complete the electro-stimulation system.

As with the preceding implementations, the analyte complexes are formed at the attached probes by liquid flows, following which a sustained drying gas stream dries the enclosed complexes before reading through the window.

The duration of flow of the drying gas stream through the cassette is typically in excess of one quarter of a minute, and in the case of arrays extending transversely to the direction of flow, usually about one half minute or more. The inlet pressure and flow volume of the drying gas stream and its temperature, as well as the duration of its flow, are chosen for enhancing efficiency of photo-emission from the analyte complexes. The precise values will depend upon the characteristics of the particular cassette and assay involved, as such factors as flow resistance and the relationship of the flow path to the complexes to be dried can affect the results. By simple trials, suitable values can readily be determined for any given cassette construction and assay type.

A number of embodiments of the invention have been described. Nevertheless, it will be understood that various modifications may be made without departing from the spirit and scope of the invention. Accordingly, other embodiments are within the scope of the following claims.

1. The method of conducting an assay by employing a cassette which encloses a surface to which at least one probe is attached, the surface associated with a liquid passage that enables liquid flow over the probe,
the assay being of the type in which one or more liquids flowing over the probe produce at the probe a bound analyte complex that has a constituent that is capable of emitting light for detection by an external detector after the light passes from the analyte complex through a window of the cassette,
wherein, after formation of the analyte complex within the cassette, the method of conducting the assay includes:
(a) by liquid flow, removing liquid resident at the analyte complex on the enclosed surface and
(b) forcing a stream of drying gas to flow into the cassette, over the enclosed surface and out of the cassette during a drying interval under conditions that substantially volatilize and remove residue of the liquid associated with the analyte complex, and performing the detection on the dried complex enclosed within the cassette.

2. The method of claim 1 conducted in a manner to form and dry an array of said complexes at a corresponding array of surface-bound probes enclosed within the cassette, and performing the detection on the array of complexes within the cassette.

3. The method of claim 2 in which at least some of the analyte complexes in the array are on probes distributed, transversely to the direction of liquid flow, upon a width-wise extended surface, the passage is correspondingly wide, constructed to produce a uniform liquid flow over the array, and the drying gas is introduced through a relatively narrow channel and spreads to flow through the liquid passage over the array.

4. The method of claim 3 in which the extended surface is a planar surface carrying a two-dimensional array of probes and corresponding analyte complexes exposed to the liquid and drying gas flows.

5. The method of claim 2 in which the probes of the array are arranged in a sequence in a line in a flow channel.

6. The method of claim 1 in which an analyte complex comprises a biological receptor probe and biological ligands carried to the probe by liquid flow.

7. The method of claim 1 in which a probe comprises oligonucleotide, peptide, polypeptide, protein or antibody, and the respective analyte comprises biomolecules that bind to the respective probe.

8. The method of claim 1 in which a complex includes a light-emissive tag associated with an analyte complex.

9. The method of claim 8 wherein an analyte complex includes a fluorescent tag.

10. The method of claim 9 in which the tag is a photo-excitable fluorescent compound that is stimulated to fluoresce by light from an external light source passing through a light-transmissive portion of the cassette.

11. The method of claim 10 in which the stimulating light and the light for detection pass through the same light-transmissive window of the cassette.

12. The method of claim 9 in which the tag is subject to electro-stimulation, an electrical pathway is provided in the cassette to the surface to which the complex is attached and the complex is stimulated by electro-stimulation via that pathway.

13. The method of claim 1 in which steps (a) and (b) are performed under automated control.

14. The method of claim 1 in which step (b) includes forcing the drying gas to flow over the enclosed surface for an interval of about one half minute or more.

15. The method of claim 5 in which step (b) includes forcing the drying gas to flow along the flow channel over complexes on the sequence of probes.

16. The method of claim 1 in which heat is delivered to the complex to promote volatilization of the liquid residue.

17. The method of claim 16 in which heat is delivered to the complex at least in part by heating the drying gas before it flows into the cassette to a temperature above ambient but below degradation temperature of the analyte complex or any associated tag.

18. The method of claim 16 in which heat is delivered to the complex at least in part by heating the surface to which the complex is bound to a temperature above ambient but below degradation temperature of the analyte complex or any associated tag.

19. The method of claim 18 comprising employing an external heater to heat the surface by thermal conduction from the exterior through a body portion of the cassette.

20. The method of claim 1 in which the surface with bound complex is a microscope slide or a microscope slide segment incorporated within the cassette.

21. The method of claim 1 in which drying gas flow into or out of the cassette flows through a device that prevents escape of liquid from the cassette.

22. The method of claim 1 in which during a liquid wash phase prior to drying, gas is caused to flow through a gas inlet channel to purge the channel of liquid contaminant.

23. The method of claim 22 in which bursts of such gas flow are introduced at spaced intervals during the wash phase.

24. An assay cassette which encloses a surface to which at least one probe is attached, the surface associated with a liquid passage that enables liquid flow over the probe, the cassette of the type enabling one or more liquids to flow over the probe to produce at the probe an analyte complex that has a constituent that is capable of emitting light that can pass through a window of the cassette for detection by an external detector, wherein, for use after formation of the analyte complex within the cassette, the cassette is constructed and arranged:
(a) to enable, by liquid flow, removal of liquid resident at the analyte complex on the enclosed surface and
(b) to enable a stream of drying gas to be forced to flow into the cassette, over the enclosed surface and out of the cassette during a drying interval under conditions that substantially volatilize and remove residue of the liquid associated with the analyte complex, to provide a dried complex within the cassette.

25. The cassette of claim 24 having an array of surface-attached probes enclosed within the cassette, the cassette constructed to enable formation and drying of an array of analyte complexes corresponding to the array of surface-attached probes and constructed to enable detection of the array of complexes within the cassette.

26. The cassette of claim 25 in which at least some of the probes are distributed transversely to the direction of liquid flow upon a width-wise extended surface, the liquid passage is correspondingly wide, constructed to produce a uniform liquid flow over the array, and the cassette is constructed to introduce the drying gas through a relatively narrow channel and to spread to flow through the liquid passage and over the array.

27. The cassette of claim 26 in which the extended surface is a planar surface carrying a two dimensional array of probes exposed to the liquid and drying gas flows.

28. The cassette of claim 25 in which the probes of the array are arranged in a sequence in a line in a flow channel.

29. The cassette of claim 24 in which a probe comprises a biological receptor probe for biological ligands carried to the probe by liquid flow.

30. The cassette of claim 29 in which a probes comprises oligonucleotide, peptide, polypeptide, protein or antibody.

31. The cassette of claim 24 containing a light-emissive tag material available to be associated with an analyte complex being formed in the cassette.

32. The cassette of claim 31 wherein the tag material is a fluorescent tag material.

33. The cassette of claim 32 in which the tag material is a photo-excitable fluorescent compound, and the cassette includes a light-transmissive portion for transmitting stimulating light from an external light source to an analyte complex that includes the tag material.

34. The cassette of claim 33 constructed to enable the stimulating light and the light for detection to pass through the same light-transmissive window of the cassette.

35. The cassette of claim 32 in which the tag material is subject to electro-stimulation, and an electrical pathway is provided in the cassette to the surface to which the probe is attached to enable a complex at the probe to be stimulated by electro-stimulation via that pathway.

36. The cassette of claim 24 constructed to enable (a) and (b) to be performed under automated control.

37. The cassette of claim 24 combined with a device adapted to force the drying gas stream to flow over the enclosed surface for an interval of about one half minute or more.

38. The cassette of claim 28 combined with a device adapted to force the drying gas stream to flow in the flow channel over a sequence of probes.

39. The cassette of claim 24 constructed to enable heat to be delivered to the complex to promote volatilization of the liquid residue.

40. The cassette of claim 39 combined with a device that heats drying gas to a temperature above ambient but below degradation temperature of the analyte complex or any associated tag.

41. The cassette of claim 39 constructed to enable heat to be delivered to the complex at last in part by heating the surface to which the complex is bound to a temperature above ambient but below degradation temperature of the analyte complex or any associated tag.

42. The cassette of claim 39 adapted for use with an external heater that heats by thermal conduction from the exterior through a body portion of the cassette.

43. The cassette of claim 24 in which the surface to which the probe is bound is a microscope slide or microscope slide segment incorporated within the cassette.

44. The cassette of claim 24 in which drying gas flow into or out of the cassette is arranged to flow through a device that prevents escape of liquid from the cassette.

45. The cassette of claim 24 combined with a control system constructed to produce gas flow during a liquid wash phase for removal of liquid contaminant.

46. The cassette of claim 45 constructed to produce bursts of the gas flow at intervals during the wash phase.

47. The assay cassette of claim 24 having a common passage for introducing liquid and drying gas flows over the surface to which a probe is attached, the cassette having a bubble removal system to which at least some of the liquids are exposed before reaching the common passage, there being multiple connections to the common passage substantially upstream of the surface with attached probe but downstream of the bubble removal system, the connections including an inlet for liquid flow from the bubble removal system and another inlet arranged to receive the drying gas stream.

48. The assay cassette of claim 47 in which there is a widening transition section between the common passage and the surface, the surface bearing an array of probes.

49. The assay cassette of claim 47 in which the bubble removal system comprises a buoyancy chamber through which liquids flow.

50. The assay cassette of claim 49 in which all of the liquids of the assay are forced to flow through a buoyancy chamber.

51. The assay cassette of claim 47 having storage volumes on board the cassette for all liquids employed in the assay.

52. The assay cassette of claim 24 combined with an air pump for producing the stream of drying air and a liquid pumping system for the liquids of the assay.

53. The assay cassette of claim 52 in which the liquid pumping system is a liquid diaphragm pumping system.

54. The cassette of claim 24 in which, the surface enclosed within the cassette supports immobilized binding domains of protein.

55. The cassette of claim 24 in which, the surface enclosed within the cassette supports immobilized binding domains of genomic nature, (oligonucleotides, SNPs, segments of genes, or other genetic material).

56. The cassette of claim 24 in which, the surface enclosed within the cassette supports immobilized binding domains of cells or cell lysate.

57. The cassette of claim 24 in which, the surface enclosed within the cassette supports immobilized binding peptides.

58. The cassette of claim 24 in which the surface enclosed within the cassette supports immobilized binding ligands comprising small organic molecules having molecular weight between about 500 to about 6000 Daltons.

59. The cassette of claim 24 in which the surface enclosed within the cassette supports immobilized binding ligands comprising small organic molecules comprising steroids, peptides or expression regulators comprising RNA primers or siRNAs.

60. An external reader station for an assay cassette which includes a system for causing liquid flows that cause formation of a light-emitting complex on a surface enclosed by the cassette and a detector for detecting light emitted from the complex that passes through a window of the cassette, the reader station including a source of pressurized drying gas and a control constructed to automatically produce a sustained stream of drying gas into and through the cassette, to dry the complex before light detection.

61. The external reader station of claim 60 constructed to automatically introduce one or more bursts of gas flow during a controlled wash phase for the cassette.

62. The external reader station of claim 60 including at least one heater for providing heat to the complex to promote drying.

63. The external reader station of claim 62 in which a heater is arranged to heat the drying gas before it enters the cassette.

64. The external reader station of claim 62 in which a heater is arranged to heat a surface of the cassette that is in heat transfers relation to the complex or to drying gas flowing to the complex.

65. The external reader station of claim 64 constructed, for drying, to heat a surface of the cassette to temperature above that employed during formation of the complex.

66. The external reader station of claim 60 including an air pump for producing the stream of drying air and a liquid pumping system for the liquids of the assay.

67. The external reader station of claim 66 in which the liquid pumping system comprises a linear actuator constructed and arranged to deflect a liquid pumping diaphragm of the cassette.
